(19) United States
(12) Patent Application Publication
(10) Pub. No.: US 2019/0299191 A1
(43) Pub. Date: Oct. 3, 2019

(54) Nanoporous Matrix and Use Thereof

(71) Applicants: Commissariat a l'Energie Atomique et aux Energies Alternatives, Paris (FR); Centre National de la Recherche Scientifique, Paris (FR)

(72) Inventors: Thu-Hoa Tran-Thi, Montrouge (FR); Ludovic Caillat, Orly (FR)

(73) Assignees: Commissariat a l'Energie Atomique et aux Energies Alternatives, Paris (FR); Centre National de la Recherche Scientifique, Paris (FR)

(21) Appl. No.: 16/316,244

(22) PCT Filed: Jul. 7, 2017
(86) PCT No.: PCT/FR2017/051868

§ 371 Date: Jan. 8, 2019

(30) Foreign Application Priority Data
Jul. 8, 2016 (FR) 16 56600

(51) Int. Cl. B01J 20/28; B01D 53/66; G01N 31/22; B01J 20/26; B01J 20/30; G01N 33/00

(52) U.S. Cl. CPC B01J 20/28047; B01D 53/66; B01J 20/28064; B01J 20/28083; B01J 20/2808; G01N 31/225; B01D 2257/106; B01J 20/30; G01N 33/0039; B01D 2258/06; B01J 2220/49; B01D 2253/106; B01J 20/262

(57) ABSTRACT

The invention relates to a nanoporous polyalkoxysilane sol-gel matrix and to a process for producing such a nanoporous polyalkoxysilane sol-gel matrix containing indigo carmine, wherein said process comprises the following steps: •a) synthesizing a gel from tetramethoxysilane or from a mixture of tetramethoxysilane and another organosilicon precursor chosen from phenyltrimethoxysilane, phenyltriethoxysilane, a fluoroalkyltrimethoxysilane, a fluoroalkyltriethoxysilane, a chloroalkylmethoxysilane, a chloroalkylethoxysilane, an aminopropyltriethoxysilane and mixtures thereof, the synthesis being carried out in an aqueous medium in the presence of a polar organic solvent and of the indigo carmine at a temperature ranging from 20 to 70° C., •b) drying the gel obtained in step a) so as to obtain a nanoporous polyalkoxysilane sol-gel matrix containing indigo carmine.

The invention relates to a process for preparing novel nanoporous matrices, to the nanoporous matrices thus obtained and to their use thereof, especially as ozone-abatement filter and ozone sensor.

The worrying effects of atmospheric pollution on health have led public authorities to implement increasingly stringent standards on pollutant emissions. Among all the pollutants, ozone represents a major problem when its concentration in the atmosphere exceeds the warning threshold of 75 ppb (180 μg·m⁻³) over one hour.

However, in certain confined spaces such as the cabin of a car, in aeroplanes [1] or in photocopying centers equipped with laser printers and photocopiers [2], the ozone concentration may reach very much higher values, largely superior to 100 ppb. Ozone is extremely harmful to the lungs, the kidneys, the brain and the eyes; it is especially the cause of respiratory problems such as asthma attacks in sensitive individuals. According to the WHO, it is a problem when its concentration in the atmosphere increases beyond a threshold of 50 ppb (i.e. 120 μg·m⁻³) per day for 8 hours.

Eradication of this harmful pollutant in interior air and especially in confined public or work spaces and the real-time monitoring of ozone are major challenges for protecting populations.

The filters most commonly used for trapping ozone are molecular filters based on active charcoal or alumina impregnated with potassium permanganate (KMnO₄). “Rigid” molecular filters are composed of active charcoal (1.25 kg/filter) protected upstream with folded prefilters (glass fiber medium, from the company Camfil). Active charcoals in granular form (diameter >2.5 mm) are manufactured from coconut and have a specific surface area of 800 m²·g⁻¹. These filters have an efficiency with respect to O₃ of the order of 58% to 79% and may have service lives of several years, but, however, with an efficiency which gradually lowers over time at and above the first six months [3].

Despite good yields and good durability, active charcoals have drawbacks. For high relative humidities of the order of 70%, the trapping efficiency decreases greatly due to the competing adsorption between water molecules in vapor form and ozone. Moreover, there is no means for controlling the saturation of active charcoals, these charcoals being known for leaching pollutants once they are saturated.

Another known ozone filter is potassium iodide (KI), which reacts with O₃ to produce either I₂ and I₃⁻ in humid medium, or KIO₃ in dry medium. However, KI reacts with all oxidizing agents and release of I₂ into the ambient air is not recommended.

Other catalytic systems are also used for destroying ozone. Many precious metals and metal oxides have been used to this effect [4]. Metal oxides, which are less expensive than precious metals, have been the subject of numerous studies. A comparison of the efficiency of metal oxides gives: MnO₂ (42%)>Co₃O₄ (39%)>NiO (35%)>Fe₂O₃ (24%)>Ag₂O (21%)>Cr₂O₃ (18%)>CeO₂ (11%)>MgO (8%)>V₂O₅ (8%)>CuO (5%)>MoO₃ (4%) [4]. These catalytic systems must, however, function at high temperature (200 to 400° C.) for good efficiency and are difficult to use continuously for months or even years. Consequently, they are rather used in certain aircraft with functioning that lasts only for the duration of the voyage. Moreover, catalytic systems are not ozone-specific.

At the present time, among all these filters, active charcoals remain the best candidates in terms of quality/price ratio and durability. However, active charcoals do not trap only O₃. They trap all kinds of volatile compounds present in the atmosphere and it is impossible to know the state of saturation of an active charcoal and the start of leaching of the harmful compounds.

Recently, indigo, in powder form or in thin film form deposited on semiconductor-based sensors, has been used as a specific filter for discriminating O₃ and NO₂ [5]. Indigo has often been used for making chemical O₃ sensors [6, 7]. In the latter cases, it is found either dissolved in a polymer [6], or in porous matrices by impregnation as a solution [7] for the detection of low contents of O₃. In all the cases cited, in powder form, thin film form, or doped porous matrix form, the specific surface areas for adsorption are too small for application as a filter.

Impregnation of active charcoals with indigo is not possible, since the efficiency of active charcoals lies in their very small pore sizes. Now, excessively small pore sizes do not make it possible to sequester the indigo molecules. Covering the outer surfaces of charcoal grains with indigo would provide only a small surface area for adsorption and consequently reduced and non-durable efficiency.

Moreover, impregnating active charcoals with potassium iodide, which could penetrate the pores of the active charcoal, might have the effect of leaching I₂ when the active charcoal is saturated. Furthermore, in this case, it is not always possible to know the state of saturation of the filter.

The aim of the present invention is to overcome the problems encountered with the O₃ filters or the pollution abatement systems marketed or described in the literature. With regard to active charcoal, these problems are especially the lack of specificity, the absence of warning on saturation, the leaching of pollutants on saturation and interference with the humidity of the air. In the case of potassium iodide powder, it is especially a lack of specific surface area for absorption and the leaching of toxic compounds (I₂). The lack of specific surface area is also encountered with porous matrices impregnated with indigo in solution. Finally, catalytic systems require a functioning at high temperature for non-specific abatement of pollutants.

The aim of the present invention is also to propose a simple and readily industrializable method for producing ozone-specific filters endowed with a saturation warning function.

Besides the trapping of ozone with filters, measuring the ozone concentration in the air is a major challenge for protecting populations. As regards measurements, several transduction principles such as mass spectrometry, chromatography or ultraviolet absorption spectrophotometry are nowadays commercially exploited for detecting ozone. The ozone analyzers generally used in the measuring stations of the Associations Agréés de Surveillance pour la Qualité de l'Air (AASQA [air quality monitoring authority]) are based on ultraviolet absorption spectrophotometry. This method is based on the direct measurement of the absorbance of ozone at 254 nm. These systems are very efficient, but also very bulky and very expensive, which reduces their use for on-site measurements where portability of the devices is required.

“Real-time” measurement has thus given rise to numerous developments of sensors based on various principles [8]. For the measurement of ozone in the atmosphere in exterior or interior air, our attention will be focused only on low-cost colorimetric sensors, functioning either in active or passive mode and capable of measuring ppb levels of O₃.

In patent U.S. Pat. No. 4,859,607 of Aug. 22, 1989 [9], Lambert et al. proposed a colorimetric sensor based on an azine of 3-methyl-2-benzothiazoline acetone mixed in 1:4 proportion with 2-phenylphenol. The mixture dissolved in the acetone is deposited on a solid substrate. In the presence of ozone, the mixture becomes violet-red. Although the intensity of the color does not vary linearly with the ozone concentration, these authors propose to use it as a passive ozone detector for ozone contents from 100 ppb to one ppm. This colorimetric sensor is ozone-selective and does not react with O₂, NO₂, SO₂, Br₂ or I₂, but gives a yellow color with Cl₂.

The dye that is the most popular in the literature remains indigo, which was initially used for dyeing fabrics. In 2004, Franklin et al. produced ozone sensors using cellulose paper impregnated with carmine indigo for measuring ozone in exterior air [10]. Since cellulose paper is opaque, these authors measured the change in color by reflectance and found that the method is less reliable than nitrite-based passive sensors. They attributed this result to the existence of interferences in the exterior air, without, however, identifying their nature.

In 2005, Marcy et al. proposed the use of various indigo-based dyes for the selective measurement of ozone [11]. These authors dissolved these compounds in a copolymer of polydimethylsiloxane and polycarbonate and deposited this mixture onto a transparent solid substrate. The ˜580 nm thick film is permeable to O₃ by virtue of the presence of the polydimethylsiloxane. Analysis of the decline in indigo absorbance at 663 nm as a function of time and of the O₃ concentration and in the presence of various potential interfering agents, NO₂ and Cl₂, enabled these authors to conclude that the sensor showed good selectivity. The response of the O₃ sensor in dynamic detection mode (1 L·min⁻¹ and relative humidity <4%) is 0.1118 ppm⁻¹·s⁻¹ and the detection limit, of 30 ppb, still remains high for measuring air quality.

In 2007, the Japanese company NTT proposed a colorimetric ozone sensor which combined carmine indigo and a porous material serving as support, the glass Vycor 7930. This glass has pores with 6 nm diameter and has a specific surface area for adsorption of 250 m²·g⁻¹ [12, 13]. The authors report that one of the problems in the preparation of the sensor is the impregnation of the substrate. Specifically, carmine indigo does not penetrate the pores of the glass Vycor unless the impregnation solution is at acidic pH<5. However, at acidic pH, protonated carmine indigo is not stable in solution. However, these authors mentioned that carmine indigo can remain stable (at least six months) in the pores of Vycor once the solvent has been evaporated off. With this sensor functioning in passive mode, the authors give a detection limit of 3 ppb for 1 hour of exposure. NTT nevertheless identified a major interference of this sensor which has sensitivity to UV radiation that is an inconvenience for its use in an external environment.

To overcome this problem of sensitivity to UV radiation, NTT proposed to incorporate a “UV-absorbing” compound and to use paper as substrate for making the ozone sensor [14]. The novel sensor proposed is a paper soaked in a first stage in an acidified (citric acid) solution of carmine indigo containing a thickener (glycerol), and then soaked a second time in an acetone solution containing a UV absorber (2-hydroxy-4-methoxybenzophenone-5-sulfonic acid, or 2-hydroxy-4-methoxybenzophenone-5-sodium sulfonate, or ferulic acid (4-hydroxy-3-methoxycinnamic acid)) and then dried for the use under consideration.

However, it is known from the literature that benzophenone derivatives, which are often used as UV radiation absorbers, themselves react with ozone [15]. Among these derivatives, 2,2′-dihydroxy-4-methoxybenzophenone is the one that is the most reactive toward ozone and produces many degradation products. Thus, in the presence of benzophenone derivatives, the ozone contents measured might be undervalued. Similarly, the reactivity of ozone toward substituted aromatic compounds is also known [16]. Thus, the use of ferulic acid might also contribute toward giving a false ozone measurement.

The aim of the present invention is also to overcome the problems encountered with the indigo-based O₃ sensors sold or described in the literature. The present invention is directed toward overcoming all or some of the drawbacks identified above, especially by avoiding the impregnation doping method.

The inventors have, to their credit, discovered, very unexpectedly and after extensive research, that it is possible to prepare nanoporous sol-gel matrices in which the pore size distribution is chosen so as both to allow ozone to pass and to sequester the probe molecule, giving the matrix both a function of filtration of air polluted with ozone and a warning function on saturation of the filter and a possibility of quantitative monitoring of the ozone present in the atmosphere.

A sol-gel matrix is a material obtained via a sol-gel process which consists in using as precursors metal alkoxides of formula M(OR)ₓR′ₙ₋ₓ in which M is a metal, especially silicon, R is an alkyl group and R′ is a group bearing one or more functions with n=4 and x possibly ranging between 2 and 4. In the presence of water, the alkoxy groups (OR) are hydrolyzed to silanol groups (Si—OH). These groups condense forming siloxane bonds (Si—O—Si—). Small particles generally less than 1 μm in size form, and aggregate to form lumps which remain in suspension without precipitating, forming a sol. The increase of the lumps and their condensation increases the viscosity of the medium, which gels. A porous solid material is obtained by drying the gel, with expulsion of the solvent outside the polymer network formed (syneresis).

One subject of the invention thus relates to a process for preparing a nanoporous polyalkoxysilane sol-gel matrix containing carmine indigo, said process comprising the following steps:
a) synthesis of a gel from tetramethoxysilane or from a mixture of tetramethoxysilane and of another organosilicon precursor chosen from phenyltrimethoxysilane, phenyltriethoxysilane, a fluoroalkyltrimethoxysilane, a fluoroalkyltriethoxysilane, a chloroalkylmethoxysilane, a chloroalkylethoxysilane, an aminopropyltriethoxysilane, and mixtures thereof, the synthesis being performed in aqueous medium in the presence of a polar organic solvent and of carmine indigo at a temperature ranging from 20 to 70° C.,
b) drying of the gel obtained in step a) so as to obtain a nanoporous sol-gel matrix containing carmine indigo.

The nanoporous polyalkoxysilane sol-gel matrices thus obtained have a proportion of micropores of greater than or equal to 10%, preferably greater than or equal to 20%, 30%, 40%, 50%, 60% or 80%. These micropores prevent the carmine indigo molecules from leaving. Ozone, for its part, can readily diffuse into the porous network, unlike large-sized molecules such as monocyclic aromatic compounds. When ozone comes into contact with the carmine indigo, it reacts therewith by electrophilic addition to the unsaturated C—C bond to give isatin or isatoic anhydride as main reaction products. This reaction is accompanied by a color change, carmine indigo being dark blue and the reaction products yellow. Thus, the nanoporous matrix according to the invention gradually changes color from dark blue via green to yellow when it is exposed to ozone, for example ozone present in the interior or exterior ambient air.

The term “micropores” means herein pores with a diameter of less than 2 nm. In general, the micropore diameter is from 0.8 to 2 nm. In the present invention, the micropore diameter is advantageously centered about 1.1±0.1 nm, i.e. at least 40% to 60% of the micropores have a diameter of about 1.1±0.1 nm.

The nanoporous sol-gel matrices according to the invention advantageously have a proportion of micropores of from 10% to 100%, preferably from 20% to 90%, more preferentially from 30% to 80%, and a proportion of mesopores of from 0% to 90%, preferably from 10% to 80%, more preferentially from 20% to 70%. Even more preferentially, they have a proportion of micropores of from 50% to 70% and a proportion of mesopores of from 50% to 30%.

A person skilled in the art will know how to choose the ad hoc proportions of the micropores/mesopores as a function of the intended application and of its constraints. Thus, for the filtering function, the objective is to trap as much O₃ as possible, while at the same time ensuring a long service life of the filter and while avoiding any leaching. High proportions of micropores preferentially greater than 50% and more preferentially greater than 80% will thus be favored. For the sensor function, the principle of which is the color change (which is proportional to the ozone content), the main aim is to rapidly measure the ozone content in the atmosphere. To do this, rapid diffusion of the ozone into the matrix must be enabled so as to obtain a rapid color change. High proportions of mesopores preferentially greater than 30% and more preferentially greater than 60% will then be favored. Advantageously, a proportion of micropores of 70% will not be exceeded for the sensor function. The nanoporous sol-gel matrices according to the invention also have a specific surface area of from 550±50 m²·g⁻¹ to 890±80 m²·g⁻¹, preferably from 650±70 m²·g⁻¹ to 890±80 m²·g⁻¹ and even more preferentially from 750±70 m²·g⁻¹ to 890±80 m²·g⁻¹.

The specific surface area and the pore size distribution are determined by analysis of the liquid nitrogen adsorption-desorption isotherm with the DFT (density functional theory) model.

The synthesis of the gel in step a) of the process according to the invention is advantageously a one-pot synthesis, i.e. it is performed in a single step with trimethoxysilane or the mixture of trimethoxysilane and the other organosilicon precursor, and carmine indigo in the presence of the polar solvent and water.

The synthesis of the gel in step a) is advantageously performed using tetramethoxysilane or a mixture of tetramethoxysilane and of another organosilicon precursor chosen from phenyltrimethoxysilane (PhTMOS), phenyltriethoxysilane (PhTEOS), a fluoroalkyltrimethoxysilane, a chloroalkylmethoxysilane, an aminopropyltriethoxysilane, and mixtures thereof, preferably from phenyltrimethoxysilane (PhTMOS), phenyltriethoxysilane (PhTEOS), a fluoropropyltrimethoxysilane, a chloropropylmethoxysilane, an aminopropyltriethoxysilane, and mixtures thereof.

In one embodiment, the synthesis of the gel is performed using tetramethoxysilane. In another embodiment, the synthesis of the gel is performed using a mixture of tetramethoxysilane and of another organosilicon precursor chosen from phenyltrimethoxysilane, phenyltriethoxysilane, a fluoroalkyltrimethoxysilane, a fluoroalkyltriethoxysilane, a chloroalkyltrimethoxysilane, a chloroalkyltriethoxysilane, an aminopropyltriethoxysilane, and mixtures thereof, preferably from a fluoropropyltrimethoxysilane, a fluoropropyltriethoxysilane, a chloropropyltrimethoxysilane, a chloropropyltriethoxysilane, an aminopropyltriethoxysilane, and mixtures thereof, preferably from phenyltrimethoxysilane, phenyltriethoxysilane, a fluoropropyltrimethoxysilane, a chloropropyltrimethoxysilane and an aminopropyltriethoxysilane, more preferably from phenyltrimethoxysilane, phenyltriethoxysilane, trimethoxy(3,3,3-trifluoropropyl)silane (3FTMOS), (3-chloropropyl)trimethoxysilane (3CITMOS) and (3-aminopropyl)triethoxysilane.

During the use of a mixture of tetramethoxysilane and of another organosilicon precursor, the molar proportions of tetramethoxysilane/other organosilicon precursor may range between 0.97/0.03 and 0.6/0.4, preferably between 0.9/0.1 and 0.7/0.3.

Specifically, tetramethoxysilane-based sol-gel matrices absorb in the UV range between 200 and 350 nm, which gives them a protective effect against UV radiation necessary for the stability of carmine indigo. This effect is further enhanced by adding phenyltrimethoxysilane, phenyltriethoxysilane, a fluoroalkyltrimethoxysilane, a chloroalkyltrimethoxysilane, an aminopropyltrimethoxysilane or a mixture thereof. Furthermore, among the compounds used as UV radiation shields, the phenyl groups [7] are the least reactive toward ozone.

The provision of phenyl groups and of long carbon and fluoro chains moreover increases the hydrophobicity of the matrix, which reduces the interference by water vapor. Thus, in one particular embodiment, the gel from step a) is prepared from a mixture of tetramethoxysilane and of another organosilicon precursor chosen from phenyltrimethoxysilane, phenyltriethoxysilane, a fluoroalkyltrimethoxysilane, a chloroalkyltrimethoxysilane, and mixtures thereof, preferably from phenyltrimethoxysilane, phenyltriethoxysilane, and a fluoroalkyltrimethoxysilane, preferably from phenyltrimethoxysilane, phenyltriethoxysilane and a fluoropropyltrimethoxysilane, more preferably from phenyltrimethoxysilane, phenyltriethoxysilane and trimethoxy(3,3,3-trifluoropropyl)silane.

The provision of a phenyl group, of chloropropyltrimethoxysilane and of a fluoroalkyltrimethoxysilane also makes it possible to increase the number of micropores (diameter <20 Å) relative to the mesopores (20<diameter <50 Å) and to improve the distribution of the micropores so as to further promote the selective trapping of small molecules such as ozone. Thus, in a particularly advantageous embodiment, the gel from step a) is prepared from a mixture of tetramethoxysilane and of phenyltrimethoxysilane or phenyltriethoxysilane.

The polar organic solvent used during the synthesis of the gel of step a) is advantageously a protic organic solvent, preferably a C1 to C6 aliphatic alcohol, more preferentially methanol or ethanol and even more preferentially methanol. The molar proportions of polar organic solvent and of water may range, respectively, between 4 and 10, preferably between 4 and 7 and more preferentially between 4 and 5.

The preparation of the gel of step a) is advantageously performed with 10⁻⁵ mol·L⁻¹ to 10⁻¹ mol·L⁻¹, preferably 2×10⁻⁴ mol·L⁻¹ to 5×10⁻² mol·L⁻¹ and even more preferentially 5×10⁻⁵ mol·L⁻¹ to 3×10⁻² mol·L⁻¹ of carmine indigo. Below 10⁻⁴ mol·L⁻¹, the nanoporous matrix will not contain enough carmine indigo to efficiently trap the ozone over a long period, and above 10⁻¹ mol·L⁻¹, the limitation is the solubility of the carmine indigo. With these starting concentrations, a nanoporous matrix with a carmine indigo content of from 10⁻⁴ mol·dm³ to 1 mol·dm³, preferably from 2×10⁻³ mol·dm³ to 5×10⁻¹ mol·dm³ and even more preferentially 5×10⁻⁴ mol·dm³ to 3×10⁻¹ mol·dm³ is obtained on conclusion of step b) of the process according to the invention.

In one embodiment, the preparation of the gel in step a) is advantageously performed with 10⁻⁴ to 10⁻¹ mol·L⁻¹, preferably 10⁻³ to 5×10⁻² mol·L⁻¹ and even more preferentially 10⁻² to 3×10⁻² mol·L⁻¹ of carmine indigo. With these starting concentrations, a nanoporous matrix with a carmine indigo content of from 10⁻³ to 1 mol·dm³, preferably from 10⁻² to 5×10⁻¹ mol·dm³ and even more preferentially 10⁻¹ mol·dm³ to 3×10⁻¹ mol·dm³ is obtained on conclusion of step b) of the process according to the invention.

In another embodiment, the preparation of the gel in step a) is advantageously performed with 10-5 to 2×10⁻⁴ mol·L⁻¹, preferably 2×10⁻⁵ to 1.5×10⁻⁴ mol·L⁻¹ and even more preferentially 5×10⁻⁵ to 10⁻⁴ mol·L⁻¹ of carmine indigo. With these starting concentrations, a nanoporous matrix with a carmine indigo content of from 10⁻⁴ to 2×10⁻³ mol·dm³, preferably from 2×10⁻⁴ to 1.5×10⁻³ mol·dm³ and even more preferentially 5×10⁻⁴ to 10⁻³ mol·dm³ is obtained on conclusion of step b) of the process according to the invention.

The preparation of the gel in step a) is advantageously performed at a temperature of from 20 to 70° C. The use of a high temperature makes it possible to accelerate the hydrolysis and the condensation of the organosilicon precursors. For example, heating of the reaction mixture (sol) at 60° C. makes it possible to accelerate the gelation by a factor of 6 to 7 relative to preparation of the gel at room temperature (20-25° C.).

The preparation of the gel is advantageously performed by first mixing the organosilicon precursor(s) with the polar organic solvent, followed by adding an aqueous solution of carmine indigo. The mixture thus obtained is mixed to obtain a sol. This sol is then poured into one or more molds and left to stand to obtain a gel. This gel is finally dried by evaporating off the water and the organic solvent to obtain the nanoporous sol-gel matrix containing carmine indigo according to the invention. The evaporation of the water and the solvent for the drying of the matrix may be performed according to any method known to those skilled in the art. The drying may be performed, for example, at room temperature or at a higher temperature, especially from 40 to 80° C. Preferably, the drying is performed at a temperature ranging from room temperature (about 20° C.) to 50° C. Advantageously, the drying is performed under an inert atmosphere.

The invention also relates to the nanoporous polyalkoxysilane sol-gel matrix containing carmine indigo which may be obtained via the process in accordance with the invention per se. Such a matrix is a polyalkoxysilane sol-gel matrix obtained from tetramethoxysilane or from a mixture of tetramethoxysilane and of another organosilicon precursor and is especially characterized in that it has a specific surface area of between 650±60 and 890±80 m²·g⁻¹, especially between 750±70 and 890±80 m²·g⁻¹, and a proportion of micropores of greater than 30%, preferably greater than 40% and even more preferentially greater than 60%. Advantageously, this matrix has a content of carmine indigo of from 10⁻³ to 10⁻² mol·dm³, preferably from 10⁻² to 5×10⁻² mol·dm³ and even more preferentially from 10⁻¹ mol·dm³ to 3×10⁻¹ mol·dm³.

In one embodiment, the sol-gel matrix according to the invention has a proportion of micropores of from 30% to 100% and a proportion of mesopores of from 70% to 0%, preferably a proportion of micropores of from 40% to 90% and a proportion of mesopores of from 60% to 10%, more preferentially a proportion of micropores of from 50% to 70% and a proportion of mesopores of from 50% to 30%. Advantageously, the matrix of this embodiment has a specific surface area of between 550±50 and 890±80 m²·g⁻¹, especially between 650±70 and 890±80 m²·g⁻¹ and more preferentially from 750±70 to 890±80 m²·g⁻¹. In this embodiment, the content of carmine indigo is advantageously from 10⁻³ to 1 mol·dm³, preferably from 10⁻² to 5×10⁻¹ mol·dm³ and even more preferentially from 10⁻¹ mol·dm³ to 3×10⁻¹ mol·dm³.

In another embodiment, the proportion of micropores is from 10% to 80%, preferably from 20% to 60% and more preferentially from 30% to 40% and the proportion of mesopores is from 20% to 90%, preferably from 40% to 80% and more preferentially from 60% to 70%. Advantageously, the matrix of this embodiment has a specific surface area of between 550±50 and 890±80 m²·g⁻¹, especially between 650±70 and 890±80 m²·g⁻¹ and more preferentially from 750±70 to 890±80 m²·g⁻¹. In this embodiment, the content of carmine indigo is advantageously from 10⁻⁴ to 2×10⁻³ mol·dm³, preferably from 2×10⁻⁴ to 1.5×10⁻³ mol·dm³ and even more preferentially from 5×10⁻⁴ mol·dm³ to 10⁻³ mol·dm³. By virtue of their particular physicochemical characteristics described above, the nanoporous polyalkoxysilane sol-gel matrices containing carmine indigo according to the invention are capable of selectively trapping ozone without having the drawbacks of leaching encountered with active charcoal. Furthermore, the colorimetric reaction between ozone and carmine indigo allows visual monitoring of the trapping of ozone. When the matrix becomes yellow, this indicates that the matrix is saturated and ozone can no longer be retained in the micropores of the matrix.

A subject of the invention is thus also the use of a nanoporous polyalkoxysilane sol-gel matrix containing carmine indigo according to the invention for selectively trapping ozone present in the air. Since the sol-gel matrix protects the carmine indigo against UV radiation, the matrix can be used for both interior air and exterior air.

In one aspect of the invention, the nanoporous polyalkoxysilane sol-gel matrix containing carmine indigo according to the invention is used as an air filter for ozone abatement. The invention thus also relates to an ozone abatement filter comprising a nanoporous polyalkoxysilane sol-gel matrix containing carmine indigo according to the invention and also to a process for filtering air by trapping the ozone present in the air in the micropores of a nanoporous polyalkoxysilane sol-gel matrix according to the invention by placing said matrix in contact with the air so as to sequester the ozone present in the air in the pores of said matrix. As described above, ozone reacts with the carmine indigo enclosed in the micropores of the matrix, which induces a color change from dark blue via green to yellow, the yellow color indicating saturation of the filter.

For use as an air filter for ozone abatement, the nanoporous polyalkoxysilane sol-gel matrix according to the invention advantageously has a carmine indigo content of from 10⁻³ to 1 mol·dm³, preferably from 10⁻² to 5×10⁻¹ mol·dm³ and even more preferentially from 10⁻¹ mol·dm³ to 3×10⁻¹ mol·dm³.

Furthermore, the nanoporous polyalkoxysilane sol-gel matrix used as an air filter for ozone abatement has a proportion of micropores of from 30% to 100%, preferably from 40% to 90% and more preferentially from 50% to 80% and the proportion of mesopores is from 0% to 70%, preferably from 10% to 60% and more preferentially from 20% to 50%. Advantageously, the matrix of this embodiment also has a specific surface area of between 550±50 and 890±80 m²·g⁻¹, especially between 650±70 and 890±80 m²·g⁻¹ and more preferentially from 750±70 to 890±80 m²·g⁻¹. In one embodiment, the matrices according to the invention are used in combination with active charcoal filters. This embodiment is particularly advantageous in the case of matrices according to the invention containing phenyl and/or fluoro groups, given that the increased hydrophobicity of these matrices makes it possible to improve the trapping of ozone in a humid atmosphere. The use of a matrix according to the invention downstream of an active charcoal filter makes it possible to indicate the saturation of said filter by means of the colorimetric reaction between ozone and carmine indigo.

In one aspect of the invention, the nanoporous polyalkoxysilane sol-gel matrix containing carmine indigo according to the invention is used in the detection of ozone present in the air. The invention thus also relates to an ozone sensor comprising a nanoporous polyalkoxysilane sol-gel matrix containing carmine indigo according to the invention and also to a process for detecting ozone present in the air, the process comprising a step of placing a nanoporous polyalkoxysilane sol-gel matrix containing carmine indigo according to the invention in contact with air so as to sequester the ozone present in the air in the pores of said matrix and a step of detecting the presence of ozone by means of the colorimetric reaction between ozone and carmine indigo. A color change of the matrix indicates the presence of ozone. This color change may be observed by the naked eye or using a spectrophotometer. The use of a spectrophotometer has the advantage of allowing quantification of the ozone present in the air. Detection using a spectrophotometer is based on the absorbance of carmine indigo at 617 nm. Since the nanoporous matrices according to the invention are transparent, this measurement may be taken both in transmission mode or in reflectance mode.

For use as an ozone sensor, the nanoporous polyalkoxysilane sol-gel matrix according to the invention advantageously has a carmine indigo content of from 10⁻⁴ to 2×10⁻³ mol·dm³, preferably from 2×10⁻⁴ to 1.5×10⁻³ mol·dm³ and even more preferentially from 5×10⁻⁴ mol·dm³ to 10⁻³ mol·dm³.

Furthermore, the nanoporous polyalkoxysilane sol-gel matrix used as ozone sensor has a proportion of micropores of from 10% to 80%, preferably from 20% to 60% and more preferentially from 30% to 40% and the proportion of mesopores is from 20% to 90%, preferably from 40% to 80% and more preferentially from 60% to 70%. Advantageously, the matrix of this embodiment has a specific surface area of between 550±50 and 890±80 m²·g⁻¹, especially between 650±70 and 890±80 m²·g⁻¹ and more preferentially from 750±70 to 890±80 m²·g⁻¹. Naturally, other embodiments of the invention could be envisaged by a person skilled in the art without, however, departing from the scope of the invention defined by the claims hereinbelow.

Non-limiting examples of implementation of the invention are described below.

### FIGURES

FIG. 1: Perspective view of an exposure cell for performing spectrophotometry measurements on rays transmitted by a nanoporous matrix when subjected to radiation from a lamp in the UV-visible range, the cell having a fluidic circuit allowing the matrix to be exposed to a controlled flow rate of air.

FIG. 2: View in cross section of the exposure cell according to FIG. 3, illustrating in detail the internal fluid circuit, and also the optical assembly.

FIG. 3: A: Spectral evolution of the indigo-doped sensor of example 1 as a function of the time of exposure to a gaseous mixture containing 50 ppb of O₃ (stream=200 mL·min⁻¹, RH=5%, T=22° C.), B: calibration curve for the sensor established using the monoexponential kinetics of decline of the absorbance at 617 nm as a function of time.

FIG. 4: A: Spectral variation of the indigo-doped sensor of example 1 as a function of time during exposure to gaseous mixtures of ozone (200 ppb) at 50% RH and 22° C. (stream=260 mL·min⁻¹). B: kinetics of decline of the absorbance of the sensor at 617 nm as a function of time. The rate of disappearance of the band is given by the slope at the origin of the curve.

### EXAMPLES

I. Preparation and Analysis of Nanoporous Sol-Gel Matrices

Analysis of the liquid nitrogen adsorption-desorption isotherm (77 K) with the DFT (density functional theory) model used for determining the specific surface area and the pore diameter was performed with the Autosorb 1 machine from Quantachrome.

### Example 1: TMOS Matrix Doped with Carmine Indigo

Reagents: carmine indigo (Sigma-Aldrich, CAS number 860-22-0, molar mass=466.35 g/mol), TMOS (CAS number: 681-84-5, molar mass=152.2 g/mol and density d=1.023 mg/L), methanol (CH₃OH, molar mass=32.04 g/mol, purity 98%). Plastic mold 12*12 cm in size and multi-wells 16*10*4 mm in size.

Procedure for 100 mL of sol: 38.89 mL of TMOS and 42.29 mL of methanol are poured into a round-bottomed flask and mixed with magnetic stirring for 1 minute. 18.82 mL of an aqueous carmine indigo solution at 0.15 mol·L⁻¹ are added to the mixture. The mixture is stirred for 1 hour at room temperature in the tightly sealed flask. The solution is poured into a plastic mold placed in a desiccator, which is covered hermetically with an aluminum membrane. When gelation takes place, the aluminum membrane is replaced with a porous film. Drying is continued in the same desiccator until the solvents have completely evaporated off and the monolith has shrunken to stable final dimensions.

After drying, sol-gel blocks in the form of dark blue parallelepipeds close to 6*5*2 mm in size are obtained. The indigo content in the nanoporous matrix is 0.28 mol·dm³.

The specific surface area for adsorption of the material obtained by analysis of the liquid nitrogen adsorption-desorption isotherm with the DFT (density functional theory) model is 890±80 m²·g⁻¹. The proportion of micropores is 84% and the diameter is centered around 1.0 nm. With these pore sizes, the carmine indigo molecules cannot leave the matrix. Ozone can readily diffuse into the porous network, unlike large-sized molecules such as monocyclic aromatic compounds.

### Example 2: TMOS/PhTMOS Matrix Doped with Carmine Indigo

Reagents: carmine indigo (Sigma-Aldrich, CAS number 860-22-0, molar mass=466.35 g·mol⁻¹), TMOS (CAS number: 681-84-5, molar mass=152.2 g·mol⁻¹ and density d=1.023 mg·cm⁻³), PhTMOS (CAS number: 2996-92-1, molar mass=198.29 g·mol⁻¹ and density d=1.062 g·cm⁻³), methanol (CH₃OH, molar mass=32.04 g·mol⁻¹, density=0.792 g·cm⁻³, purity 98%). Plastic multi-well mold (16*10*4 mm).

Procedure for 100 mL of sol: 34.623 mL of TMOS and 4.926 mL of PhTMOS are placed in 41.837 mL of methanol in a round-bottomed flask and mixed with magnetic stirring for 1 minute. 18.615 mL of an aqueous carmine indigo solution at 0.15 mol·L⁻¹ are added to the mixture. The mixture is stirred for 1 hour at room temperature in the tightly sealed flask. The solution is poured into a plastic mold placed in a desiccator, which is covered hermetically with an aluminum membrane. When gelation takes place, the aluminum membrane is replaced with a porous film. Drying is continued in the same desiccator until the solvents have completely evaporated off and the monolith has shrunken to stable final dimensions.

In the present example, the molar proportions of the reagents are TMOS/PhTMOS/MeOH/H₂O: 0.9/0.1/4/4. The indigo content in the nanoporous matrix is 0.28 mol·dm³.

The specific surface area for adsorption of the material obtained by analysis of the liquid nitrogen adsorption-desorption isotherm with the DFT (density functional theory) model is 800±50 m²·g⁻¹. The proportion of micropores is 40% and the diameter is centered around 11 nm. The diameter of the mesopores is centered around 25 Å. With these pore sizes and the tortuosity of the porous network, the carmine indigo molecules cannot leave the matrix. Ozone can readily diffuse into the porous network, unlike large-sized molecules such as monocyclic aromatic compounds.

### Example 3: TMOS/PhTEOS Matrix Doped with Carmine Indigo

Reagents: carmine indigo (Sigma-Aldrich, CAS number 860-22-0, molar mass=466.35 g·mol⁻¹), TMOS (CAS number: 681-84-5, molar mass=152.2 g·mol⁻¹ and density d=1.023 mg·cm⁻³), PhTEOS (CAS number: 780-69-8, molar mass=240.37 g·mol⁻¹ and density=0.996 g·cm⁻³), methanol (CH₃OH, molar mass=32.04 g·mol⁻¹, density=0.792 g·cm⁻³, purity 98%). Plastic multi-well mold (16*10*4 mm).

Procedure for 100 mL of sol: 26.365 mL of TMOS and 14.252 mL of PhTEOS are placed in 41.097 mL of methanol in a round-bottomed flask and mixed with magnetic stirring for 1 minute. 18.285 mL of an aqueous carmine indigo solution at 0.15 mol·L⁻¹ are added to the mixture. The mixture is stirred for 1 hour at room temperature in the tightly sealed flask. The solution is poured into a plastic mold placed in a desiccator, which is covered hermetically with an aluminum membrane. When gelation takes place, the aluminum membrane is replaced with a porous film. Drying is continued in the same desiccator until the solvents have completely evaporated off and the monolith has shrunken to stable final dimensions.

In the present example, the molar proportions of the reagents are TMOS/PhTEOS/MeOH/H₂O: 0.75/0.25/4.3/4.3. The indigo content in the nanoporous matrix is 0.28 mol·dm³.

The specific surface area for adsorption of the material obtained by analysis of the liquid nitrogen adsorption-desorption isotherm with the DFT (density functional theory) model is 630±50 m²·g⁻¹. The proportion of micropores is 70% and the diameter is centered around 1.1 nm. The diameter of the mesopores is centered around 22 Å. With these pore sizes and the tortuosity of the porous network, the carmine indigo molecules cannot leave the matrix. Ozone can readily diffuse into the porous network, unlike large-sized molecules such as monocyclic aromatic compounds.

### Example 4: TMOS/PhTEOS Matrix Doped with Carmine Indigo

Reagents: carmine indigo (Sigma-Aldrich, CAS number 860-22-0, molar mass=466.35 g·mol⁻¹), TMOS (CAS number: 681-84-5, molar mass=152.2 g·mol⁻¹ and density d=1.023 mg·cm⁻³), PhTEOS (CAS number: 780-69-8, molar mass=240.37 g·mol⁻¹ and density=0.996 g·cm⁻³), methanol (CH₃OH, molar mass=32.04 g·mol⁻¹, density=0.792 g·cm⁻³, purity 98%). Plastic multi-well mold (16*10*4 mm).

Procedure for 100 mL of sol: 31.90 mL of TMOS and 9.13 mL of PhTEOS are placed in 40.81 mL of methanol in a round-bottomed flask and mixed with magnetic stirring for 1 minute. 18.16 mL of an aqueous carmine indigo solution at 0.15 mol·L⁻¹ are added to the mixture. The mixture is stirred for 1 hour at room temperature in the tightly sealed flask. The solution is poured into a plastic mold placed in a desiccator, which is covered hermetically with an aluminum membrane. When gelation takes place, the aluminum membrane is replaced with a porous film. Drying is continued in the same desiccator until the solvents have completely evaporated off and the monolith has shrunken to stable final dimensions.

In the present example, the molar proportions of the reagents are TMOS/PhTEOS/MeOH/H₂O: 0.85/0.15/4/4. The indigo content in the nanoporous matrix is 0.28 mol·dm³.

The specific surface area for adsorption of the material obtained by analysis of the liquid nitrogen adsorption-desorption isotherm with the DFT (density functional theory) model is 560±50 m²·g⁻¹. The proportion of micropores is 40% and the diameter is centered around 1.1 nm. The diameter of the mesopores is centered around 24 Å. With these pore sizes and the tortuosity of the porous network, the carmine indigo molecules cannot leave the matrix. Ozone can readily diffuse into the porous network, unlike large-sized molecules such as monocyclic aromatic compounds.

### Example 5: TMOS/3CITMOS Matrix Doped with Carmine Indigo

Reagents: carmine indigo (Sigma-Aldrich, CAS number 860-22-0, molar mass=466.35 g·mol⁻¹), TMOS (CAS number: 681-84-5, molar mass=152.2 g·mol⁻¹ and density d=1.023 mg·cm⁻³), 3ClTMOS (CAS number: 2530-87-2, purity 97%, molar mass=198.72 g·mol⁻¹ and density=1.09 g·cm⁻³), methanol (CH₃OH, molar mass=32.04 g·mol⁻¹, density=0.792 g·cm⁻³, purity 98%). Plastic multi-well mold (16*10*4 mm).

Procedure for 100 mL of sol: 30.488 mL of TMOS and 9.627 mL of 3ClTMOS are placed in 41.445 mL of methanol in a round-bottomed flask and mixed with magnetic stirring for 1 minute. 18.44 mL of an aqueous carmine indigo solution at 0.15 mol·L⁻¹ are added to the mixture. The mixture is stirred for 1 hour at room temperature in the tightly sealed flask. The solution is poured into a plastic mold placed in a desiccator, which is covered hermetically with an aluminum membrane. When gelation takes place, the aluminum membrane is replaced with a porous film. Drying is continued in the same desiccator until the solvents have completely evaporated off and the monolith has shrunken to stable final dimensions.

In the present example, the molar proportions of the reagents are TMOS/3ClTMOS/MeOH/H₂O: 0.8/0.2/4/4. The indigo content in the nanoporous matrix is 0.28 mol·dm⁻³.

### Example 6: TMOS/3FTMOS Matrix Doped with Carmine Indigo

Reagents: carmine indigo (Sigma-Aldrich, CAS number 860-22-0, molar mass=466.35 g·mol⁻¹), TMOS (CAS number: 681-84-5, purity 97%, molar mass=152.2 g·mol⁻¹ and density d=1.142 mg·cm⁻³), 3FTMOS (CAS number: 429-60-7, molar mass=218.25 g·mol⁻¹ and density=0.996 g·cm⁻³), methanol (CH₃OH, molar mass=32.04 g·mol⁻¹, density=0.792 g·cm⁻³, purity 98%). Plastic multi-well mold (16*10*4 mm).

Procedure for 100 mL of sol: 25.854 mL of TMOS and 16.108 mL of 3FTMOS are placed in 40.167 mL of methanol in a round-bottomed flask and mixed with magnetic stirring for 1 minute. 17.872 mL of an aqueous carmine indigo solution at 0.15 mol·L⁻¹ are added to the mixture. The mixture is stirred for 1 hour at room temperature in the tightly sealed flask. The solution is poured into a plastic mold placed in a desiccator, which is covered hermetically with an aluminum membrane. When gelation takes place, the aluminum membrane is replaced with a porous film. Drying is continued in the same desiccator until the solvents have completely evaporated off and the monolith has shrunken to stable final dimensions.

In the present example, the molar proportions of the reagents are TMOS/3FTMOS/MeOH/H₂O: 0.7/0.3/4/4. The indigo content in the nanoporous matrix is 0.28 mol·dm³.

The specific surface area for adsorption of the material obtained by analysis of the liquid nitrogen adsorption-desorption isotherm with the DFT (density functional theory) model is 460±60 m²·g⁻¹. The proportion of micropores is 40% and the diameter is centered around 1.6 nm. With these pore sizes, the carmine indigo molecules cannot leave the matrix. Ozone can readily diffuse into the porous network, unlike large-sized molecules such as monocyclic aromatic compounds.

### Example 7: TMOS/APTES Matrix Doped with Carmine Indigo

Reagents: carmine indigo (Sigma-Aldrich, CAS number 860-22-0, molar mass=466.35 g·mol⁻¹), TMOS (CAS number: 681-84-5, purity 97%, molar mass=152.2 g·mol⁻¹ and density d=1.142 mg·cm⁻³), APTES (CAS number: 919-30-2, molar mass=208.33 g·mol⁻¹ and density=0.933 g·cm⁻³), methanol (CH₃OH, molar mass=32.04 g·mol⁻¹, density=0.792 g·cm⁻³, purity 98%). Plastic multi-well mold (16*10*4 mm).

Procedure for 100 mL of sol: 33.91 mL of TMOS and 1.574 mL of APTES are placed in 45.582 mL of methanol maintained at low temperature (−25° C.) in a round-bottomed flask and mixed with magnetic stirring for 2 minutes. 16.934 mL of an aqueous carmine indigo solution at 0.15 mol·L⁻¹ are added to the mixture. The mixture is stirred for 2 minutes and the solution is poured into a plastic mold and then placed in a desiccator, which is covered hermetically with an aluminum membrane. When gelation takes place, the aluminum membrane is replaced with a porous film. Drying is continued in the same desiccator until the solvents have completely evaporated off and the monolith has shrunken to stable final dimensions.

In the present example, the molar proportions of the reagents are TMOS/APTES/MeOH/H₂O: 0.97/0.03/5/4. The indigo content in the nanoporous matrix is 0.28 mol·dm³.

The specific surface area for adsorption of the material obtained by analysis of the liquid nitrogen adsorption-desorption isotherm with the DFT (density functional theory) model is 820±70 m²·g⁻¹. The proportion of micropores is 20% and the diameter is centered around 17 Å. The proportion of mesopores is 80% and the diameter is centered around 27 Å.

II. Ozone Trapping Tests

The trapping tests of the indigo-doped matrices exposed to O₃ were performed to quantify the trapping efficiency and thus to demonstrate their utility as ozone abatement filters. The matrices in parallepipedal form obtained in Examples 1 to 6 were ground coarsely and screened (passed through two screens) to obtain millimeter-sized granules.

The filters were prepared in the form of plastic syringes (6 mL volume) equipped with two end pieces, one of which is connected via a tube to the gaseous mixture generator. The outlet end piece of the syringe is connected to a tube which goes into a fume cupboard. These syringes were filled with the various granules and exposed to a mixture of air containing ozone.

The analysis bench includes mass flowmeters and a humidity injection system to allow exposure of the filters as a function of the flow rate of the gaseous mixture at various humidities. Ozone is generated with an O₃ generator and its concentration is measured upstream and downstream of the filter by means of an O₃ analyzer.

The flow rate of the stream upstream of the cartridge is 2 L/min and the flow rate downstream was measured for each cartridge so as to check that the pressure loss is approximately the same for all the cartridges. The degree of trapping, τ, is measured for each type of filter as a function of the exposure parameters. It is defined according to:

$$τ = \frac{[ O {3}_{upstream} ] - [ O {3}_{downstream} ]}{[ O {3}_{upstream} ]} 100 \%$$

The ozone concentration ([O3ᵤₚₛₜᵣₑₐₘ]) chosen, ˜40 ppb, corresponds to a mean value of the ozone content in the exterior air. The effect of the humidity of the stream of gaseous mixture (% RH=50 and 72) was also studied. The trapping performance qualities were compared for exposure times ranging from 1 minute to 480 hours and up to 20 days for certain tests. Moreover, a comparison of the trapping performance was performed with a commercial ozone trap based on KI and a trap based on active charcoal.

The data obtained are collated in the tables below.

TABLE 1
Compared performances of trapping of O₃
at low concentration for various materials
Upstream stream = 2 L/min, [O₃]ᵤₚₛₜᵣₑₐₘ = 42 ± 3 ppb, RH = 50%

| | Degree of trapping | | |
|---|---|---|---|
| | Mat 1 | Mat 2 | Commercial |
| Time (h) | (1.79 g) | (1.73 g) | KI (1.5 g) |
| 1 | 100% | 100% | 80% |
| 24 | 94% | 100% | 61% |
| 48 | 82% | 100% | 48% |
| 72 | 72% | 100% | |
| 168 | 59% | 94% | |
| 384 | 30% | 64% | |
| 480 | 22% | 55% | |
| Downstream flow rate | 1.32 | 1.22 | 1.44 |
| (L/min) | | | |

The materials Mat 1 (Example 1) and Mat 2 (Example 2) both have small pores, but Mat 2 is more hydrophobic, which explains its better performance and service life. Comparison with the commercial product containing KI shows the superiority of the sol-gel materials which simultaneously combines a large specific surface area for adsorption suitable for trapping O₃.

Tests at high relative humidity, RH=72%, were also performed to compare the efficiency of O₃ trapping under conditions in which the water vapor concentration is very high and might contribute toward clogging the pores of the filter and reducing its efficiency.

TABLE 2
Compared performances of trapping of O₃ at high humidity of an
indigo-doped nanoporous filter with an active charcoal filter
Upstream stream = 2 L/min, RH = 72%

Degree of trapping

| | | Mat 2 | CA RBAA3 |
|---|---|---|---|
| Exposure time (h) | [O₃] (ppb) | (1.79 g) | (1.75 g) |
| 24 | 43 ± 5 | 100% | 100% |
| 48 | 43 ± 5 | 100% | 90% |
| 72 | 43 ± 5 | 98% | 89% |
| 96 | 43 ± 5 | 92% | 79% |
| Downstream flow rate | | 1.27 | 1.16 |
| (L/min) | | | |

The active charcoal chosen (Norit RBAA-3 rods, Fluka) has very small pores (pore diameter <11 Å) and a specific surface area for adsorption of 960 m²·g⁻¹. The data collated in table 2 show that Mat 2 traps ozone better than active charcoal at high humidity.

Tests were also performed at a very high concentration of ozone.

TABLE 3
Compared performances of trapping of O₃ at a high concentration
of O₃ for various indigo-doped materials
Upstream stream = 2 L/min, [O₃]ᵤₚₛₜᵣₑₐₘ = 475 ± 10 ppb, RH = 50%

| | Degree of trapping | | | | |
|---|---|---|---|---|---|
| | Mat 1 | Mat 2 | Mat 3 | Mat 6 | Mat 7 |
| Time (min) | (1.79 g) | (1.79 g) | (1.79 g) | (1.79 g) | (1.79 g) |
| 10 | 99.5% | 100% | 100% | 90.8% | 100% |
| 20 | 99.5% | 97.3% | 99.4% | 89.2% | 97.3% |
| 180 | 74.0% | — | 57.6% | 55.0% | 50.0% |
| 300 | 51.0% | 71.2% | 52.0% | 39.0% | 20.0% |
| 1440 | 22.0% | 26.0% | 31.0% | — | 2.0% |
| Downstream flow rate | 1.32 | 1.22 | 1.22 | 1.30 | 1.42 |
| (L/min) | | | | | |

The materials Mat 1, Mat 2 and Mat 3, which have a high proportion of micropores, have the best O₃ trapping efficiencies at high concentration of the pollutant. Mat 7 is the material which has a high percentage of mesopores and is the least efficient.

Moreover, one of the advantages of the filter based on carmine indigo is the color change that is visible to the naked eye, which makes it possible to assess its state of saturation. Specifically, the color of the filter changes gradually as ozone is trapped, from blue via green to yellow. For material 2, the filter was still not saturated after 20 days of exposure to a stream of 2 L/min with an O₃ content of 42±3 ppb, at a relative humidity of 50%.

III. Measurement of Ozone with the Matrix of Example 1

The matrix (sensor) is positioned in an exposure cell including a fluidic circuit through which passes the gas stream mixture containing ozone. The exposure cell is equipped with two optical windows through which passes the analysis light conveyed by optical fibers and originating from a UV-visible lamp. After passing through the sensor, the transmitted light is collected by an Ocean-Optics miniature spectrophotometer and the collected data are transmitted to the computer.

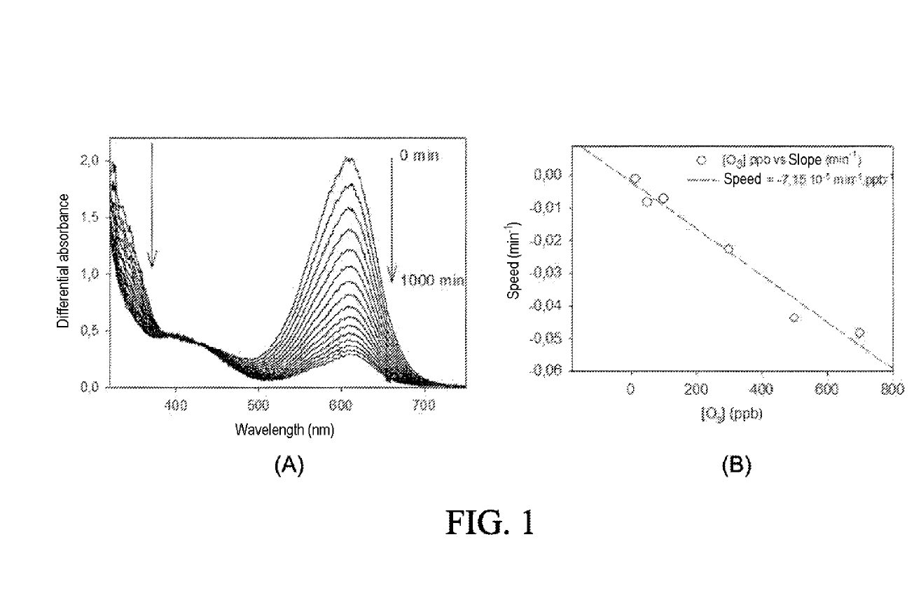

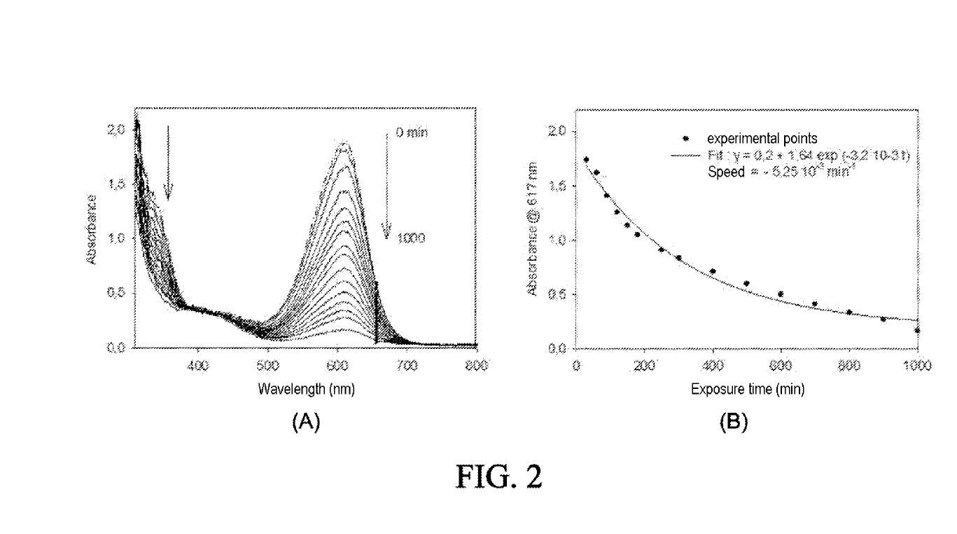

This exposure cell 1 is illustrated in FIGS. 1 and 2 and comprises a body 1 which is composed of two parts 2,3, an upper part and a lower part, respectively. The nanoporous matrix M is positioned by an operator in a cavity 30 formed on the lower part 3, when the two parts 2,3 are dismantled. This cavity 30 is then closed in a gas tight manner by assembling the upper part 2 on the lower part 3.

The optical assembly comprises a first optical window, materialized by a traversing machined channel 20 from the upper part 2, emerging in the cavity 30, and through which passes the light conveyed by the optical fibers 4 and originating from the UV-visible lamp. A second optical window, materialized by a traversing machined channel 33 from the lower part 3 allows the collection of the rays transmitted by the nanoporous matrix. The collection is performed by optical fibers 5 which convey the collected rays to the Ocean-Optics miniature spectrophotometer.

The fluidic circuit is materialized on the lower part 3 and comprises a first connection 6 connected to an air feed pipe, which emerges in a traversing machined channel 31 of the lower part 3 conveying the air originating from the pipe to the cavity 30. Another traversing machined channel 32 of the lower part 3 allows the evacuation of the gas from the cavity 30 to a second connection 7, connected to an evacuation pipe.

A spectrum is collected before exposure. During exposure, the spectra are collected every 10 minutes for 48 hours.

The gas stream containing ozone at various concentrations originates from a model 165 Megatec ozone generator. The concentration was varied from 3 to 1000 ppb. The ozone concentration was controlled beforehand with a model 49C Megatec ozone analyzer. The exposure flow rate was set at 260 mL·min⁻¹, and is controlled with a mass flowmeter. The humidity of the mixture is provided with a second stream of nitrogen humidified to 100% (by sparging in a bottle of water) with which is mixed the ozone stream.

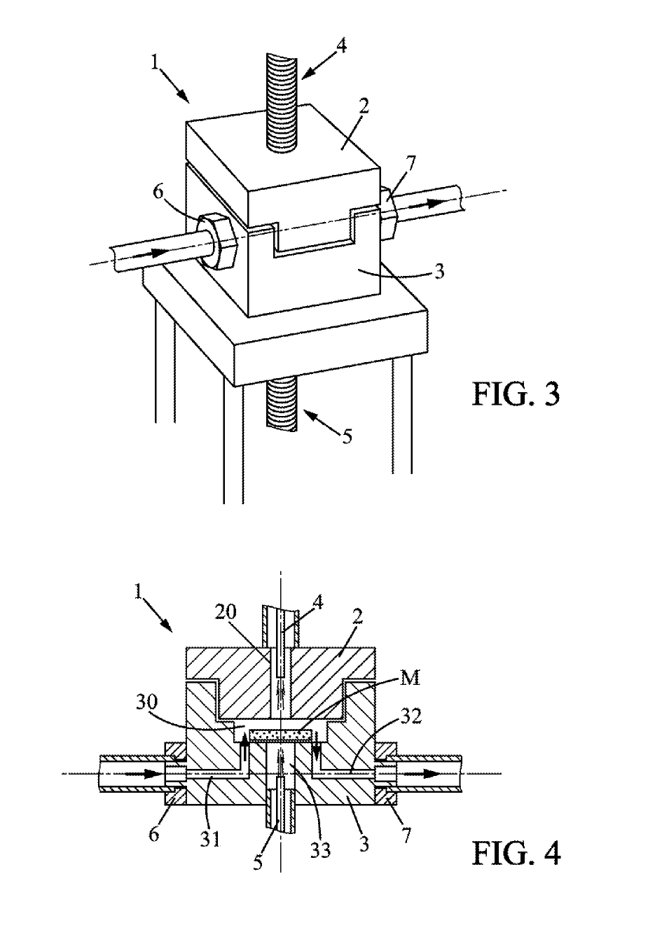

An example of spectra collected during the exposure of the matrix of Example 1 to a gaseous mixture containing 50 ppb of ozone (stream=200 mL·min⁻¹, RH=5%, T=22° C.) is shown in FIG. 3.

The decline kinetics were established from curves of variation of absorbance at 617 nm as a function of time for various ozone concentrations. The reaction rates were deduced from the monoexponential decline kinetics and reported as a function of the ozone concentration. The calibration curve thus established shows a linear variation of the rate of loss of color with the ozone concentration with a coefficient of −7.15×10⁻⁵ min⁻¹·ppb⁻¹. The detection limit is 3 ppb for an exposure of 60 minutes to a flow rate of 260 mL·min⁻¹. The sensitivity of such a sensor could be further improved by increasing the exposure flow rate of the sensor.

The color variation of the ozone sensor is also visible to the naked eye. The sensor, which is initially blue (carmine indigo alone), becomes green in the presence of yellow-colored isatin resulting from the reaction of the carmine indigo with ozone (blue+yellow=green), and then yellow at the end of exposure when all of the carmine indigo has disappeared in favor of the isatin.

IV. Measurement of Ozone with the Matrix of Example 1 in the Presence of 50% Humidity

The process was performed as in point III, but the relative humidity level of the exposure stream was 50%. The results obtained are collated in FIG. 4. It is observed that the response of the sensor is indeed sensitive and reproducible even with this high humidity level of the order of 50%.

The calibration curve established for a relative humidity of 50% shows that the response of the sensor is slower with a rate of −2.6×10⁻⁵ ppb⁻¹·min⁻¹, i.e. 2.8 times slower than that obtained at 5% RH. Water interferes by decreasing the trapping of ozone in the nanoporous sensor.

### REFERENCES

[1] Clifford Weisel, Charles J. Weschler, Kris Mohan, Jose Vallarino, John D. Spengler, Ozone and Ozone Byproducts in the Cabins of Commercial Aircraft, Environ. Sci. Technol., dx.doi.org/10.1021/es3046795.
[2] P. Huré, X. Rousselin, L'émission d'ozone par les photocopieurs et imprimantes laser [Emission of ozone by laser printers and photocopiers], INRS, Ed. No. 1422, 1997.
[3] A. Ginestet, D. Pugnet, L'efficacite des filtres moléculaires pour la ventilation des batiments [The efficiency of molecular filters for the ventilation of buildings], Centre technique des industries aérauliques et thermiques, June/July 07 bimonthly.
[4] Todor Batakliev, Vladimir Georgiev, Metody Anachkov, Slavcho Rakovsky, Gennadi E. Zaikov, Ozone decomposition, Interdisciplinary Toxicology, 2014; Vol. 7(2): 47-59.
[5] J. P. Viricelle, A. Pauly, L. Mazet, J. Brunet, M. Bouvet, C. Varenne, C. Pijolat, Selectivity improvement of semi-conducting gas sensors by selective filter for atmospheric pollutants detection, Materials Science and Engineering C 26(2006) 186-195.
[6] M. Alexy, G. Voss, J. Heinze, Optochemical sensor for determining ozone based on novel soluble indigo dyes immobilised in a highly permeable polymeric film. Analytical and Bioanalytical Chemistry, 382 (2005), 1628-1641.
[7] M. Yasuko Yamada, Measurement of ambient ozone using newly developed porous glass sensor, Sensors and Actuators B 126 (2007) 485-491.
[8] G. Kiriakidis, J. Kortidis, K. Moschovis, D. Dovinos, On the Road to Inexpensive sub-ppb Room temperature Ozone Sensing, Sensor Lett. Vol. 6, No. 6 (2008), 812-816.
[9] J. L. Lambert, Y. C. Chiang, J. V. Paukstelis, Colorimetric detector for ozone and method of preparation, U.S. Pat. No. 4,859,607 of 22 Aug. 1989.
[10] Anna C. Franklin et al., “Ozone Measurement in South Carolina Using Passive Sampler”, Journal of the Air & Waste Measurement Association, Vol. 54, pages 1312-1320, 2004, doi: 10.1080/10473289.2004. Ser. No. 10/470,997.
[11] M. Alexy, G. Voss, J. Heinze, Optochemical sensor for determining ozone based on novel soluble indigo dyes immobilised in a highly permeable polymeric film. Analytical and Bioanalytical Chemistry, 382 (2005), 1628-1641.
[12] M. Yasuko Yamada, Measurement of ambient ozone using newly developed porous glass sensor, Sensors and Actuators B 126 (2007) 485-491.
[13] General Information on Vycor Glass No. 7930 (Corning Glass Works, Corning, N.Y. 14830).
[14] Takashi Miwa, Yasuko Maruo, Jiro Nakamura, Tatsuya Kunioka, Seizo Sakata, Ozone detecting element, WO 2008056513 A1, 1 May 2008.
[15] Gago-Ferrero P., Demeestere K., Silvia Díaz-Cruz M., Barceló D., Ozonation and peroxone oxidation of benzophenone-3 in water: effect of operational parameters and identification of intermediate products, Sci. Total Environ. 2013, Jan. 15; 443: 209-17. doi: 10.1016/j.scitotenv.2012.10.006.
[16] Femando J. Beltran, Ozone reaction kinetics in water and waste water systems, Taylor & Francis e-library, 2005.

1. A process for preparing a nanoporous polyalkoxysilane sol-gel matrix containing carmine indigo, said process comprising the following steps:
a) synthesis of a gel from tetramethoxysilane or from a mixture of tetramethoxysilane and of another organosilicon precursor chosen from phenyltrimethoxysilane, phenyltriethoxysilane, a fluoroalkyltrimethoxysilane, a fluoroalkyltriethoxysilane, a chloroalkylmethoxysilane, a chloroalkylethoxysilane, an aminopropyltriethoxysilane, and mixtures thereof, the synthesis being performed in aqueous medium in the presence of a polar organic solvent and of carmine indigo at a temperature ranging from 20 to 70° C.,
b) drying of the gel obtained in step a) so as to obtain a nanoporous polyalkoxysilane sol-gel matrix containing carmine indigo.

2. The process as claimed in claim 1, wherein the synthesis of the gel in step a) is a one-pot synthesis.

3. The process as claimed in claim 1, wherein the synthesis of the gel in step a) is performed using tetramethoxysilane or a mixture of tetramethoxysilane and of another organosilicon precursor chosen from phenyltrimethoxysilane (PhTMOS), phenyltriethoxysilane (PhTEOS), a fluoropropyltrimethoxysilane, a fluoropropyltriethoxysilane, a chloropropylmethoxysilane, a chloropropylethoxysilane, an aminopropyltriethoxysilane, and mixtures thereof.

4. The process as claimed in claim 3, wherein the synthesis of the gel in step a) is performed using tetramethoxysilane.

5. The process as claimed in claim 3, wherein the synthesis of the gel in step a) is performed using a mixture of tetramethoxysilane and of another organosilicon precursor chosen from phenyltrimethoxysilane, phenyltriethoxysilane, a fluoropropyltrimethoxysilane, a chloropropylmethoxysilane, an aminopropyltriethoxysilane, and mixtures thereof.

6. The process as claimed in claim 5, wherein the synthesis of the gel in step a) is performed using a mixture of tetramethoxysilane and of another organosilicon precursor chosen from phenyltrimethoxysilane, phenyltriethoxysilane, trimethoxy(3,3,3-trifluoropropyl)silane, (3-chloropropyl)trimethoxysilane and (3-aminopropyl)triethoxysilane.

7. The process as claimed in claim 5, wherein the synthesis of the gel in step a) is performed using a mixture of tetramethoxysilane and of phenyltrimethoxysilane or of phenyltriethoxysilane.

8. The process as claimed in claim 3, wherein the synthesis of the gel in step a) is performed using a mixture of tetramethoxysilane and of another organosilicon precursor chosen from phenyltrimethoxysilane, phenyltriethoxysilane, a fluoroalkyltrimethoxysilane, a chloroalkyltrimethoxysilane, and mixtures thereof.

9. The process as claimed in claim 3, wherein the molar proportions of tetramethoxysilane/other organosilicon precursor are between 0.97/0.03 and 0.6/0.4.

10. The process as claimed in claim 1, wherein the polar organic solvent is methanol.

11. The process as claimed in claim 1, wherein the molar proportions of polar organic solvent and of water are, respectively, between 4 and 10.

12. A nanoporous polyalkoxysilane sol-gel matrix containing carmine indigo, obtained from tetramethoxysilane or from a mixture of tetramethoxysilane and of another organosilicon precursor chosen from phenyltrimethoxysilane, phenyltriethoxysilane, a fluoroalkyltrimethoxysilane, a fluoroalkyltriethoxysilane, a chloroalkylmethoxysilane, a chloroalkylethoxysilane, an aminopropyltriethoxysilane, and mixtures thereof, wherein it has a specific surface area of from 550±60 m²·g⁻¹ to 890±80 m²·g⁻¹ and a proportion of micropores of greater than 30%.

13-15. (canceled)

16. An ozone abatement filter comprising the nanoporous polyalkoxysilane sol-gel matrix containing carmine indigo as claimed in claim 12.

17. An ozone sensor comprising the nanoporous polyalkoxysilane sol-gel matrix containing carmine indigo as claimed in claim 12.

18. A method of selectively trapping ozone present in the air comprising contacting the air with the nanoporous polyalkoxysilane sol-gel matrix containing carmine indigo as claimed in claim 12.

19. The method of claim 18, wherein the air is filtered through the nanoporous polyalkoxysilane sol-gel matrix present in an air filter.

20. The method of claim 18, wherein ozone collected in the nanoporous polyalkoxysilane sol-gel matrix containing carmine indigo is detected to measure the presence of ozone.
